(19) United States
(12) Patent Application Publication
(10) Pub. No.: US 2026/0232999 A1
(43) Pub. Date: Aug. 13, 2026

(54) A METHOD AND APPARATUS FOR ELECTROPORATION

(71) Applicant: MIRAI MEDICAL LIMITED, Galway (IE)

(72) Inventors: Declan SODEN, Galway (IE); Sean KINSELLA, Galway (IE); Jason McNAMARA, Galway (IE); Colin FORDE, Galway (IE); Cian KERRIGAN, Galway (IE)

(73) Assignee: MIRAI MEDICAL LIMITED, Galway (IE)

(21) Appl. No.: 19/474,759

(22) PCT Filed: May 9, 2024
(86) PCT No.: PCT/EP2024/062875

§ 371 Date: Oct. 13, 2025

(30) Foreign Application Priority Data
May 12, 2023 (EP) 23173213.2

(51) Int. Cl. A61N 1/32; A61N 1/05; A61N 1/08; A61N 1/36

(52) U.S. Cl. CPC A61N 1/327; A61N 1/08; A61N 1/0509; A61N 1/36002

(57) ABSTRACT

Irreversible electroporation of mammalian cells is achieved by an electroporation apparatus comprising a controller, a drive circuit controlled by the controller to generate drive pulses, and at least two electrodes linked with the drive circuit for delivering the pulses. The controller causes the drive circuit to provide pulses in: (a) a reversible electroporation group of pulses sufficient to create/open cell pores and/or disrupt cell membranes of tissue near the electrodes, (b) an electrophoretic group of one or more pulses with lower voltages, and (c) repetition of (a) and (b) in N cycles, N having a value of 0 to 100. This achieves IRE with lower voltages and less patient discomfort.

### INTRODUCTION

The invention is in the field of electroporation.

Electroporation is the process of applying an electrical pulse of sufficient magnitude to a living cell to induce the creation of pores which transect the cell membrane. Electroporation has become established as a safe and effective clinical tool which in permeabilising the cell membrane enables the rapid passive diffusion and targeted uptake of therapeutic agents.

Reversible Electroporation (RE) is electroporation which is present for a period of time before the pores on the membrane reseal and the cell recovers. Irreversible Electroporation (IRE) is electroporation which the cell membrane is incapable of repairing after the pore formation sufficiently to perform its function of managing cell contents and consequently a process of cell death is triggered. Traditionally, Irreversible Electroporation requires the delivery of a higher voltage or a larger quantity of pulses than Reversible Electroporation.

Passage of electric current through a tissue produces Ohmic heating in accordance with Joule's law. Ohmic heating is a function of local current density and tissue resistivity. In general, when delivering Irreversible Electroporation for the purposes of tissue ablation, generation of excess heat during ablation is undesirable as heat, although an effective tissue destroyer, tends to destroy the extra-cellular matrix that supports the cells and thus healthy tissue regeneration is prevented. Thermally induced cell death can result in damage to healthy tissue structures, impede healing and risk a perforation or fistula forming in the lumen of the gastrointestinal tract or other tissue structures.

A method of inducing irreversible electroporation of tissue that causes less resistive heat generation within the target tissue is desired.

### SUMMARY OF THE INVENTION

In the following description the term V/cm means an applied voltage per centimetre spacing between the electrodes. Thus, a parameter of 100 V/cm would apply to a pair of probes 1 cm apart and which have an applied potential of 100 V across the electrodes, or to a pair of probes 2 cm apart if the applied potential is 200 V across the electrodes.

The present invention enables irreversible electroporation of tissue using pulse amplitudes lower than those currently required. It achieves this by the addition of a secondary follow-on pulse which acts to impact the cell and its membrane's response to the electroporation pulses being delivered.

We describe an electroporation apparatus comprising a controller, a drive circuit controlled by the controller to generate drive pulses, and at least two electrodes linked with the drive circuit for delivering the pulses, wherein the controller is configured to cause the drive circuit to provide pulses in:
(a) a reversible electroporation group of pulses sufficient to create/open cell pores and/or disrupt cell membranes of tissue near the electrodes,
(b) a secondary group of one or more pulses at least some of which have lower voltages than pulses of the group (a), and
(c) repetition of (a) and (b) in N cycles, N having a value of 0 to 100.

In some preferred examples, the group (b) of pulses include pulses having parameters to induce a stress on the cell membrane, enlarging the cell pores from the group (a) and to facilitate electrophoresis within the tissue, accelerating the loss of ions from the electroporated cells, reducing the capacity for recovery and triggering non-thermal cell death.

In some preferred examples, at least some of said (b) group of pulses have parameters to trigger cell death without destruction of an extracellular matrix.

In some preferred examples, the (a) group comprises a series of M bursts of pulses, each burst having a plurality of pulses.

In some preferred examples, at least some of the (a) group comprises bipolar/biphasic pulses.

In some preferred examples, at least some of the (a) group of pulses have a field strength in the range of 100 V/cm to 2500 V/cm.

In some preferred examples, at least some of the (a) group of pulses have a field strength in the range of 350 V/cm to 1500 V/cm, preferably 400 V/cm to 1300 V/cm, and more preferably 400 V/cm to 1100 V/cm.

In some preferred examples, at least some of the (a) group of pulses each have a pulse length in the range of 0.1 μs to 10 ms, preferably in the range 0.1 μs to 1 ms, more preferably in the range of 1 μs to 5 μs.

In some preferred examples, at least some of the (a) group of pulses are provided in bursts of pulses separated by time delays which are greater than time delays between individual pulses, and at least some bursts each have a duration in the range of 0.1 ms to 1000 ms, preferably in the range of 0.1 ms to 5 ms.

In some preferred examples, at least some of the (a) group of pulses are provided in bursts of pulses separated by time delays which are greater than time delays between individual pulses, and the number of bursts in each cycle is in the range of 1 to 1000, preferably in the range of 5 to 50.

In some preferred examples, the group (b) of pulses includes pulses having a field strength in the range of 1 V/cm to 100 V/cm, preferably in the range of 5 V/cm to 75 V/cm, and more preferably in the range of 10 V/cm to 50 V/cm.

In some preferred examples, at least some of the group (b) of pulses include pulses which are square wave monophasic pulses of positive or negative polarity.

In some preferred examples, at least some of the group (b) of pulses include at least one pulse having a length in the range of 10 ms to 1000 ms, preferably in the range of 100 ms to 1000 ms, more preferably in the range of 250 ms to 750 ms.

In some preferred examples, the total time for the treatment is less than one minute, preferably is in the range of 6 ms to 20 seconds.

In some preferred examples, the controller is configured to choose the value of N according to a volume of tissue to be treated.

In some preferred examples, the value of N is in the range of 1 to 100, preferably 2 to 10, more preferably 2 to 6.

In some preferred examples, the (a) group has a total energised time in the range of 5 ms to 20 ms for each cycle.

We also describe a method of treatment of tissue in a mammal, the treatment being carried out with use of a probe having at least two electrodes and a drive circuit controlled by a controller to generate drive pulses, wherein the controller causes the drive circuit to provide pulses in:
(a) a reversible electroporation group of pulses sufficient to create/open cell pores and/or disrupt cell membranes of tissue near the electrodes,
(b) a secondary group of one or more pulses, and
(c) repetition of (a) and (b) in N cycles, N having a value of 0 to 100.

In some preferred examples, the group (b) of pulses include pulses having parameters to induce a stress on the cell membrane, enlarging the cell pores from the group (a) and to facilitate electrophoresis within the tissue, accelerating the loss of ions from the electroporated cells, reducing the capacity for recovery and triggering non-thermal cell death.

In some preferred examples, said (b) group of pulses have parameters to trigger cell death without destruction of an extracellular matrix.

### DETAILED DESCRIPTION OF THE INVENTION

The invention will be more clearly understood from the following description of some embodiments thereof, given by way of example only with reference to the accompanying drawings in which:

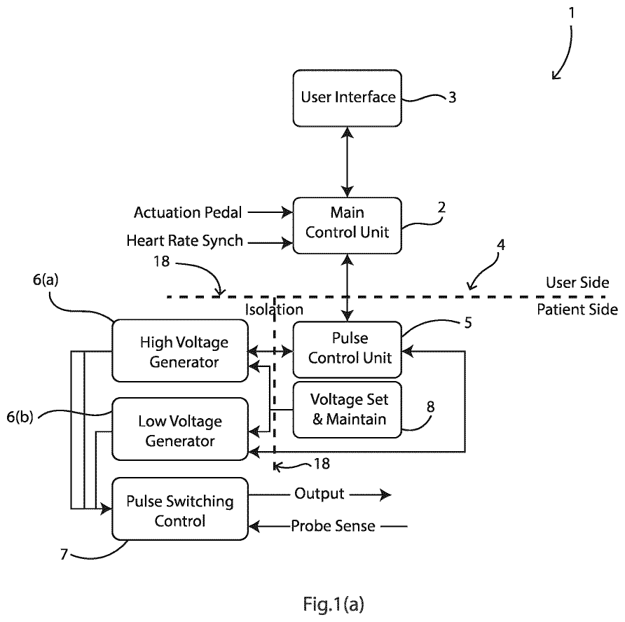

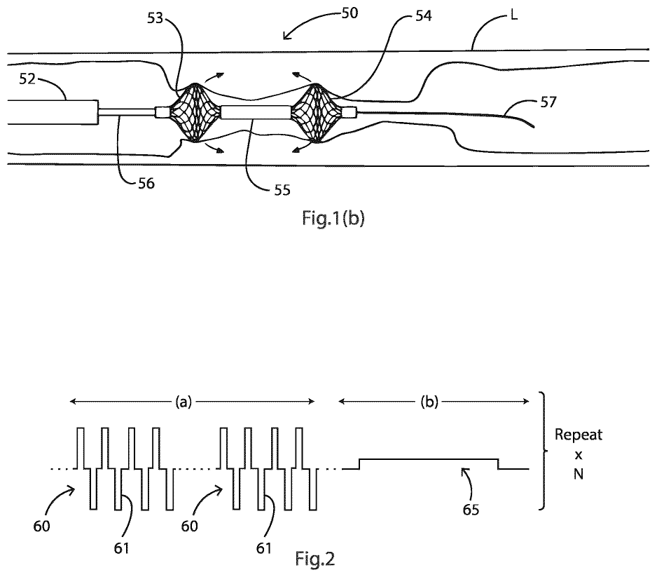

FIG. 1(a) is block diagram of an electroporation apparatus of the invention, and FIG. 1(b) shows a probe of the apparatus,

FIG. 2 is a diagrammatic plot illustrating the general manner in which the probe electrodes are driven, termed an “ERASE” drive scheme in this specification,

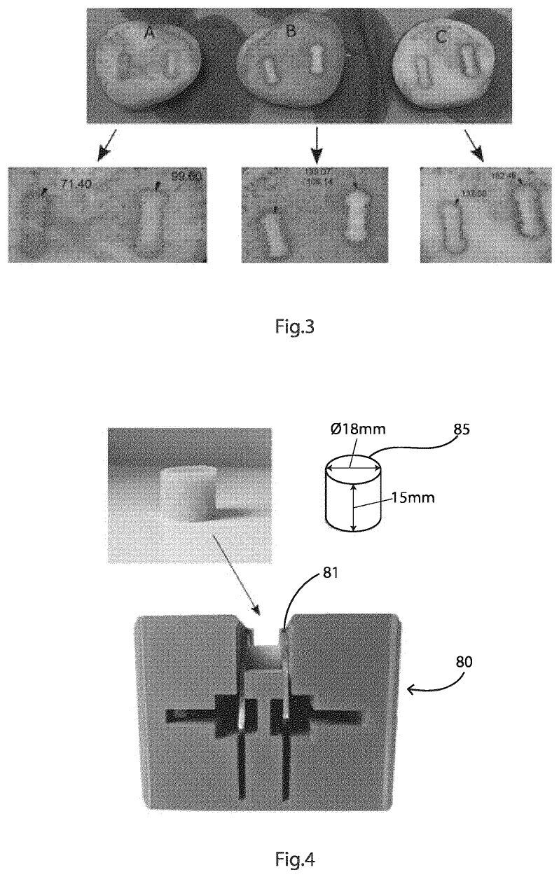

FIG. 3 is an image showing the impact of the invention on area treated using one type of electrode in a potato model,

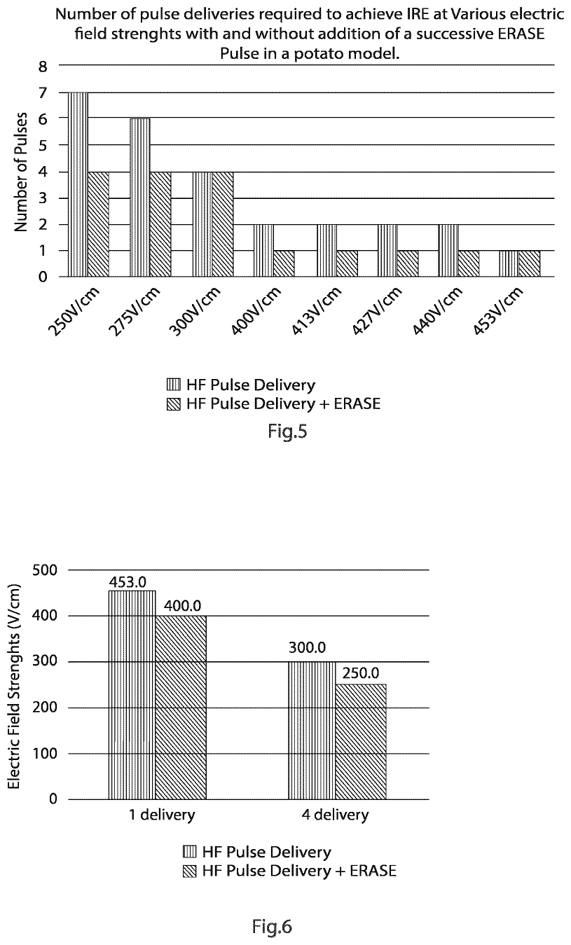

FIG. 4 shows the test article, dimensions of same and a bespoke parallel plate fixture used for reliable delivery of specific electric field strengths used to generate data supporting FIG. 5 and FIG. 6,

FIG. 5 is a clustered bar chart demonstrating the influence the ERASE drive scheme has on the number of pulses required to induce IRE in a potato model,

FIG. 6 is a clustered bar chart demonstrating the influence the ERASE protocol has on the electric field intensity required to induce IRE in a potato model,

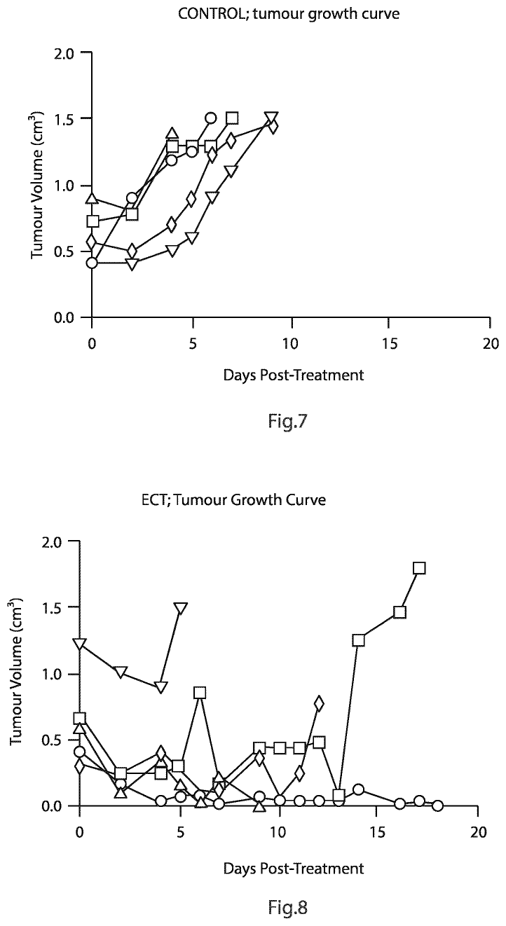

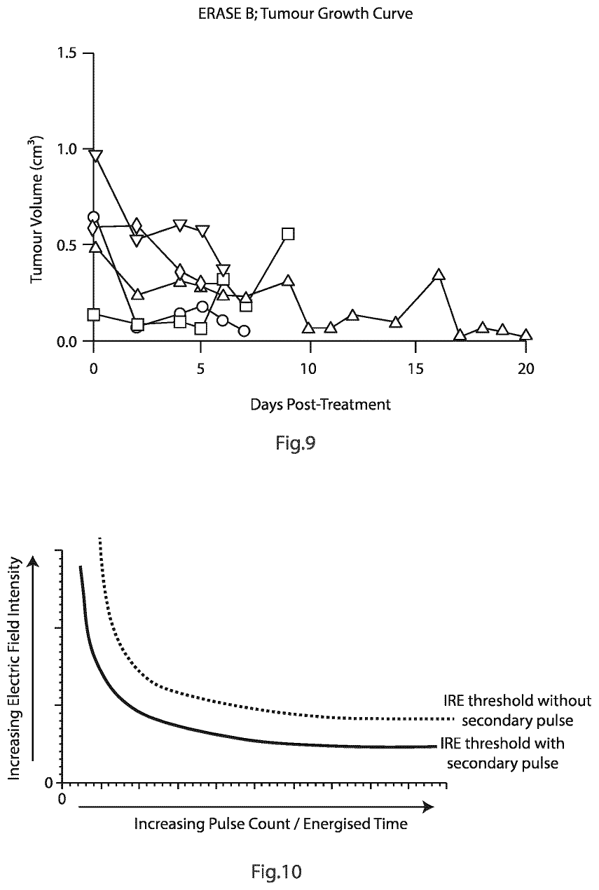

FIGS. 7 to 9 are plots showing test results obtained from in an in-vivo mouse study, in which FIG. 7 is a control set of plots, and FIGS. 8 and 9 are plots illustrating the benefits of treatment according to the invention,

FIG. 10 is an illustration of the impact of the ERASE protocol on the IRE threshold which is dimensionless as the IRE threshold is different for different tissues, and

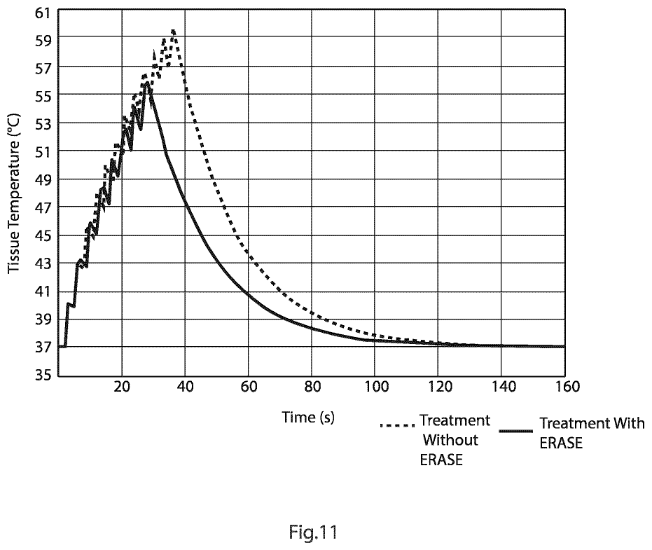

FIG. 11 is an illustration of the temperature increases seen with delivery of a group of exemplary high frequency pulses to a tissue without the use of the ERASE protocol (dashed line) and delivery of the same degree of treatment with the ERASE protocol (continuous line).

### DETAILED DESCRIPTION OF THE INVENTION

Referring to FIGS. 1(a) and (b), an electroporation apparatus 1 comprises a main controller 2 linked with a user interface 3 and with a probe drive circuit 4, all mounted in a housing 9. The controller 2 and the interface 3 provide the user-side functions, whereas the drive circuit 4 provides the pulses to a probe 50.

The drive circuit 4 comprises:
a pulse control unit 5 which is separated by opto-isolators 18 from the remainder of the drive apparatus 4, to avoid high voltages being inadvertently transferred to the low-voltage control electronics,
a voltage generator 6 with a transformer, providing a voltage across capacitors,
a pulse switching controller 7, for delivering high frequency pulses to probe electrodes 53 and 54, and
a voltage set and maintain circuit 8.

The touch screen interface 3 is operably coupled to the controller 2 which manages the drive pulse control, and the voltage, pulse duration, polarity, and orientation. This level of control is achieved via the series of control circuits in the blocks 5-8.

The drive may for example be that described in our published specification WO2021/043779, the contents of which are incorporated herein by reference. However, in the invention the drive is configured to provide pulses of a lower voltage level, although the hardware architecture may be similar.

The electroporation probe 50 (shown in FIG. 1(b)) is for delivery of electroporation treatment to cancerous and pre-cancerous regions of the gastrointestinal tract. The probe 50 comprises a sheath 52 within which there is a shaft 56 and within which there is a delivery wire 57. Two electrical conducting wires, not shown, extend within the shaft 56 for linking an electrical drive to the sets of electrodes on expanding bodies 53 and 54 separated along the longitudinal axis by an isolating spacer 55. This probe is merely one example of a probe that may use the present invention. For example, the probe may alternatively be hand-held with the electrodes protruding longitudinally for contact with target tissue.

Advantageously, the controller provides pulses which are lower in voltage in comparison to the voltage levels of the past for treatment, including treatment of cancerous tissue.

In the present invention the drive provides pulses to the electrodes 53 and 54, or other electrode arrangements, which have a relatively low voltage but are nevertheless very effective.

As shown in FIG. 2 the drive circuit is controlled by the controller to generate drive pulses for the electrodes in:
(a) a reversible electroporation group of bursts 60 (two bursts 60 in this illustration), each burst being of pulses 61 (biphasic in this example), sufficient to create/open cell pores and/or disrupt cell membranes of tissue near the electrodes,
(b) an electrophoretic-like secondary group of one or more low voltage pulses 65 (in this case monophasic), and
(c) repetition of (a) and (b) in N cycles, N having a value of 0 to 100, N being 0 meaning that there is no repetition and the stages (a) and (b) are only performed once.

The voltage magnitude required to achieve irreversible electroporation is lower than the state of the art and/or can be achieved with a lower number of high voltage pulses. The delivery of lower voltage magnitude pulses and/or less of these pulses results in lower joule heating of tissue and thus less need for consideration of the management of said heating. The secondary group of one or more pulses of an order of magnitude lower voltage than the first group do induce Joule heating in and of themselves, however this is comparatively insignificant and in aggregating all pulses and totalling Joule heating the apparatus of the invention is capable of inducing similar cell death in the target tissue at a lower overall energy delivery thus potentially providing a safer treatment, a clinical benefit for the receiver of the treatment.

In more detail, the apparatus of the invention is configured to deliver pulses to achieve the following.
(a) Electroporation, to disrupt the cell membrane and create pores:
   In mammalian tissue a field strength of >100 V/cm and <2500 V/cm but preferably in the range of 350-1500 V/cm, more preferably in the range of 400-1300 V/cm and yet more preferably in the range of 400-1100 V/cm.
   Individual Pulse length in the range of 0.1 μsec-10 ms but preferably in the range 0.1 μsec-1 ms, more preferably in the range of 0.1-5 μsec.
   Energised time per pulse burst in the range of 0.05-1000 ms but preferably in the range of 0.05-50 ms per pulse burst, more preferably in the range of 0.05-5 ms per pulse burst. Total number of pulse bursts in each cycle in the range of 1-1000 but preferably in the range of 5-50 bursts of monophasic, biphasic or sinusoidal waveforms, more preferably biphasic. Each burst is delivery of a set number of individual biphasic pulses. For example, fifty biphasic pulses of 2 μs positive and 2 μs negative, giving 200 μs energised time per burst. If there are 30 bursts in a cycle (one N value) then there is a total energized time per cycle of 6 ms. In general, it is preferred that the (a) group has a total energised time per cycle in the range of 5 ms to 20 ms before the low voltage pulse of group (b).
(b) Secondary Pulse Group:
   One or more pulses sufficient to induce a stress on the cell membrane, enlarging the pores from the first group (a) and facilitating electrophoresis within the tissue, accelerating the loss of ions from the electroporated cells, reducing the capacity for recovery and triggering non thermal cell death without destruction of the extracellular matrix:
      Voltage in the range of 1-100 V but preferably in the range of 5-75 V and yet more preferably in the range of 10-50V.
      Square wave monophasic pulse, positive or negative polarity into a monopolar or bipolar electrode. Positive or negative polarity in this sense denotes the direction a charged ion will move under the influence of the applied electric field In an electrode having just two poles there are thus two directions. For more complicated electrode designs having many poles which can act as anode or cathode it is possible that application of this electrophoretic pulse in multiple different directions may enhance the observed efficacy.
      Pulse length in the range of 10-10000 ms but preferably in the range of 100-1000 ms, more preferably in the range of 250-750 ms delivered as a single pulse or multiple pulses until the total desired pulse length is delivered.
(c) Combination and Sequence:
   Electroporation pulse group followed by secondary pulse group.
   (a) and (b) being performed from 1-100 times, preferably 2-10 times, more preferably 2-6 times.

A reduction in the electric field threshold required to create Irreversible Electroporation is observed with this drive scheme. Tissue which has undergone electroporation has higher conductivity than tissue which has not, and the degree of impedance change measured can reflect the degree of change induced in the tissue.

In the invention in various examples there is a sequence of higher voltage pulses, of shorter duration followed by a sequence of low voltage pulses (5-20× lower voltage), with longer duration (from 2 microseconds per individual pulse to >100 ms) to cause irreversible electroporation and cell death. This combination sequence is referred to as ‘ERASE’.

The pulse sequence arises from our understanding that by using a high voltage pulse burst in the reversible electroporation range, followed by a low voltage pulse burst, will firstly create pores in the cell membrane before the low voltage pulse will stress the cell membrane enlarging the pores and draw ions out of the electroporated cell and/or place adequate stress on the electroporated cell membrane to impact the ability of the cell to recover. By repeating this pulse sequence, the volume of tissue irreversibly electroporated expands.

### Example 1

We completed the following testing in a potato model, using a probe as described in our published US patent specification no. US2021/0128910, “Cutis”. This probe has two parallel linear arrays of 4 needles which act as alternative anode and cathode during delivery of pulses. Rectangular needle arrays like this are considered, in the art, to act similar to parallel plate electrodes where the volume between the two arrays is subjected to an electric field strength approximate to that which is obtained from the following simple calculation:

$$E Field between Flat Plates ( V / cm ) = \frac{Input Voltage ( V )}{Distance between Flat Plate Electrodes ( cm )}$$

The potato model is commonly used for electroporation studies since the electroporated area becomes dark, over 12 to 36 hours after delivery of pulses, the darkened area corresponding to IRE of the potato cells and thus this allows determination of the ablation boundary without specialised equipment. It is however important to note that potatoes have different, lower, thresholds for Irreversible and Reversible Electroporation than human or animal tissue. An alternative method of demonstrating the Irreversible Electroporation region in potatoes involves the use of 2,3,5-triphenyltetrazolium chloride (TTC) which is reduced by the metabolic activity of living cells or tissues to form a red coloured by-product triphenylformazan. When applied to potato tissue the IRE area of the potato appears white and the area around this which remains metabolically active appears red. In addition to this the area immediately surrounding the white area takes on an enhanced level or redness which is presumed in the art to relate to this area receiving reversible electroporation and thus having a higher uptake of the TTC in the cells resulting in a higher red intensity in this area as a greater number of TTC molecules have access to the metabolic activity within the cell.

FIG. 3 demonstrates three potatoes which have been sliced in half before treatment with the CUTIS device. On the left of each potato the electrode delivered an electric field strength 750V/cm in the form of a series of eight square wave 100 μs pulses of 300V amplitude delivered at a frequency of 5000 Hz. On the right of each potato the electrode delivered the same pulse as the left however this was followed with a secondary pulse to complete the ERASE protocol. This secondary pulse (electrophoresis, group (b)) was ten square wave 65,000 μs (65 ms) pulses of 50V amplitude delivered at a frequency of 1 Hz.

Potato A was treated once with these pulses (N=1), Potato B was treated twice with these pulses (N=2), and Potato C was treated three times with these pulses (N=3). The potatoes were immediately taken and submerged face down in a 0.5% (by weight) TTC solution. The images in FIG. 3 were obtained post 24 hours at which time the colour contrast is very well developed. These images were taken with the potatoes parallel to the plane of the camera and the camera at a height to reduce parallax error contribution. These images were then taken into a Computer Aided Design software package, Rhinoceros 7 SR15, scaled based off known dimensions, the outline of the metabolically dead region outlined and the software used to obtain area measurements for these bounded regions. These area measurements have been overlayed on the FIG. 3 and are tabulated in the following table.

| | White Area-Metabolically inactive tissue | |
|---|---|---|
| | Without ERASE (Control, left | |
| | side of each image in FIG. 3) | With ERASE |
| Potato A (1 delivery pulses) | 71.4 mm² | 99.6 mm² |
| Potato B (2 delivery pulses) | 106.1 mm² | 139.1 mm² |
| Potato C (3 delivery pulses) | 137.6 mm² | 162.5 mm² |

Results should be viewed as demonstrative of the change between those areas treated with and without ERASE as opposed to precise values as a result of the subjectivity of determining where exactly the colour changes from white to red as this is a blurry border the operator is trying to identify as opposed to a clean definitive border line. Nonetheless, it can be seen that the addition of ERASE produces a large increase in area treated: A 39%; B 39% and C 18%. Potato A with use of the ERASE protocol is almost equivalent in electroporated area demonstrated to that seen in Potato B Control. When considered that these areas are just a slice of a 3D volume treated it can be understood that the 3D volume will likely increase also in depth. It is logical that the ERASE protocol will have varying degree of addition to this treated area as with increase in the delivered dose of the pulse without ERASE there is potential you approach a degree of tissue electroporation where an ERASE pulse will have minimal additional benefit as the tissue in the relevant area has already successfully been electroporated—is thus higher in conductivity than the non electroporated tissue and a preferential path for any electrical pulse introduced.

### Example 2

The increase in treated area demonstrated in Example 1 corresponds with areas of tissue that are further from the electrodes, at a lower field strength, achieving irreversible electroporation. Testing was carried out using a flat plate test fixture wherein the field strength is one single value for a given test. The test fixturing and samples are illustrated in FIG. 4. Test samples 85 are cylinders of potato tissue of diameters ~18 mm and height 14.9-15.0 mm. These are placed between spring loaded electric plates 81 of the fixture 80 thus ensuring good electrical contact between the plates and the sample with the plates free to sit an angle if the sample has ends which are not parallel. Post test samples were sectioned with half placed in 0.5% (by weight) TTC solution and have left free to develop melanin darkening if IRE had taken place. A large number of tests were performed for different input voltages.

FIG. 5 illustrates the impact that addition of the ERASE protocol has on the number of pulse deliveries required before IRE is observed in a potato model. Pulses delivered for this testing were high frequency biphasic 2-1-2 μs pulses delivered in 30 bursts of 50 pulses per burst delivered at 10 Hz. It can be observed that for a field strength of 250 V/cm IRE was induced after just 4 pulse deliveries with ERASE versus the required 7 pulse deliveries without ERASE. It can also be observed that as the pulse strength was increased the ERASE impact had less effect. It is important to remember that these thresholds are dependant not only on the tissue being treated but also attributes of the pulse being delivered, the pulse length, type and frequency of delivery in ways that are not yet close to being fully understood in the state of the art.

FIG. 6 illustrates the impact that addition of the ERASE protocol has on the field strength required for IRE to be observed in a potato model. Pulse type was identical to that used for FIG. 5. When extrapolated to the higher voltages required in treating mammalian tissue this degree of difference in IRE threshold could be the difference between a given electrode or modality of treatment being feasible for reasons of patient tolerability or generator capability.

### Example 3

Preclinical testing has been carried out with an electroporation probe using a mouse tumour colorectal model. The electroporation probe used was designed specifically for the study in order to allow delivery of pulses to the mouse tumours through the mouse skin and works similar in principle to the CUTIS in Example 1. The electroporation probe designed has six 0.25 mm diameter sharp needles organised as two parallel linear arrays with a needle edge to edge distance of 3 mm. Needles extend 10 mm from the body of the electrode which has gripping features that allow the user to push the needles through the mouse skin atraumatically and control the depth of treatment.

In its use in this study the probe was considered to only have treated the cuboid of tissue bounded by the outer corners of the array and the needle depth, therefore in order to ensure coverage of the tumours the device was replaced in the tissue a number of times until the entire tumour volume had received treatment. Depending on tumour volume and shape this was between four and seven applications.

Mice were seeded in the flank with cancer cells. Tumour growth was monitored over a period of time with sufficient time allowed to elapse such that a majority of tumours were allowed to grow to between 0.8 to 1.2 cm³ in volume. Tumour volume was determined using the following equation:

$$Volume = \frac{1}{2} ( Length \times {Width}^{2} )$$

All mice were remeasured, sorted by tumour size and broken into groups with similar mean and standard deviation for tumour size. Five groups were studied with five mice per group:
A) Control group, no treatment.
B) Electrochemotherapy (ECT)
C) ‘ERASE’ Combination parameters×1.
D) ‘ERASE’ Combination parameters×4, and
E) IRE.

FIG. 7. Demonstrates tumour growth in the days after index for A) “Control group, No treatment”. For ethical reasons it is undesirable that tumours are allowed to exceed 1.5 cm³. As a result of the continued tumour growth due to lack of treatment all mice had been culled for humane reasons by day 9 post index.

FIG. 8. Demonstrates tumour growth in the days after index for B) “Electrochemotherapy (ECT)”. Chemotherapeutic agent Bleomycin (250IU) was injected into the centre of the tumour mass immediately before use of the electrode. The electrode delivered an electric field strength 1000V/cm in the form of a series of eight square wave 100 μs pulses of 300V amplitude delivered at a frequency of 5000 Hz. This is significantly lower in both number of pulses (8<<100) and field strength (1000V/cm<1500 V/cm) than the requirement to induce IRE in any animal tissue and thus without addition of a chemotherapeutic should have negligible effect on tumour growth. Electrochemotherapy works based on the principle that reversibly electroporated cells have greatly enhanced uptake of a directly injected or intravenously administered chemotherapeutic drug. Reversible electroporation has been demonstrated to increase the uptake of Bleomycin in the cell cytosol resulting in a corresponding increase in its cytotoxicity of 300-5000-fold (Orlowski et al. 1988, Gehl et al. 1998, Jaroszeski et al. 2000a).

FIG. 9. Demonstrates tumour growth in the days after index for D) “‘ERASE’ Combination pulse×4”, N=4. The electrode delivered for the group (a), reversible electroporation, an electric field strength 1000V/cm in the form of a series of eight square wave 100 μs pulses of 300V amplitude delivered at a frequency of 5000 Hz. This is significantly lower in both number of pulses (8<<100) and field strength (1000V/cm<1500 V/cm) than the requirement to induce IRE in animal tissue. This was followed by delivering a series of (b) group electrophoresis pulses, ten square wave 50V (low voltage) pulses of 65,000 μs (65 ms) delivered at 1 Hz. This sequence was delivered four times (N=4) at each placement of electrode. The addition of a suitably designed low voltage follow on pulse to a higher voltage pulse, capable of inducing reversible electroporation alone, shifts the electroporation from reversible to irreversible and we refer to the combination as “ERASE” or an ERASE protocol. These pre-clinical results have shown a decrease in tumour diameter post-treatment for the combination parameters which are comparable to the results obtained in the group treated with electrochemotherapy.

FIG. 10 is an illustrated graph of the impact of the ERASE protocol on the IRE threshold which is dimensionless as the IRE threshold is different for different tissues. The dashed line represents the IRE threshold without the application of an ERASE protocol and this line is shifted down and to the left by the application of ERAS. Thus far our testing in a potato model with multiple different types of preceding electroporation pulse type, high and low frequency, support this representation of its impact. In addition, preliminary testing in a murine colorectal cancer model has show efficacy of pulses at lower pulse amplitude than required for traditionally required to induce cell death.

FIG. 11 is an illustration of the temperature increases seen with delivery of a series of exemplary high frequency pulses to perfused ex-vivo liver tissue, using a parallel plate electrode with plate-to-plate distance 1 cm, without the use of the ERASE protocol and with the ERASE protocol. The dashed line represents the treatment without the use of ERASE protocol, in this example a series of twelve 1000V/cm High Frequency Biphasic Pulses are delivered with a seven second interval between deliveries. With each delivery of pulses the tissue temperature increases before decreasing somewhat before delivery of the next due heat dissipation to the surrounding tissues and environment. The tissue temperature peaks at approximately 60° C.

The solid line represents treatment with the use of the ERASE (invention) protocol, in this example eight (N=8) cycles each of the same 1000 V/cm High Frequency Biphasic 2-1-2 μs pulses, each cycle having 30 bursts of 50 pulses per burst delivered at 10 Hz (Electrophoretic, group (a)) followed by the secondary pulse (electrophoresis, group (b)) of ten square wave 65,000 μs (65 ms) pulses of 50V amplitude delivered at a frequency of 1 Hz. These were delivered with a seven second interval between commencement of each ERASE cycle.

The ERASE protocol treatment can be seen to cause a tissue temperature increase to 55° C. This 5° C. lower peak tissue temperature is sufficient to greatly decrease the degree of thermal tissue injury and its associated risks. Thermal tissue damage relates both to the temperature the tissue experiences and the length of time for which this temperature is sustained. It is considered that, in humans, tissue damage starts when the tissue temperature exceeds 43° C. and each additional degree of increase halves the time that the tissue can be maintained at a given temperature before it becomes non-viable. It can be understood thus that when tissue achieves a peak temperature of 60° C. this is significantly more damaging than achieving a peak temperature of 55° C., In addition, during its cooling towards ambient temperature the tissue will spend time at each temperature value below the peak value with undesired thermal damage being cumulative.

### Advantages

In general, it will be appreciated that the invention achieves very effective electroporation with minimal risk of patient discomfort.
Lower voltage than traditional IRE
Lower voltage than HFIRE (high frequency IRE).
Simpler generator required-reduces complexity, cost.
Quicker procedure than H-FIRE with the same ablation effects.
Removes drug
Makes clinical procedure easier.
Lower risk of pain
No muscle relaxants required
Can increase ablation zones
Non-thermal

Components of embodiments can be employed in other embodiments in a manner as would be understood by a person of ordinary skill in the art. The invention is not limited to the embodiments described but may be varied in construction and detail.

1. An electroporation apparatus comprising a controller, a drive circuit controlled by the controller to generate drive pulses, and at least two electrodes linked with the drive circuit for delivering the pulses, wherein the controller is configured to cause the drive circuit to provide pulses in:
(a) a reversible electroporation group of pulses-sufficient to create/open cell pores and/or disrupt cell membranes of tissue near the electrodes, wherein the apparatus at least some of the (a) group of pulses provide a field strength in the range of 100 V/cm to 2500 V/cm;
(b) a secondary group of one or more pulses at least some of which have lower voltages than pulses of the group (a), wherein the group (b) of pulses includes pulses having a field strength in the range of 1 V/cm to 100 V/cm; and
(c) repetition of (a) and (b) in N cycles, N having a value of 1 to 100.

2. The apparatus as claimed in claim 1, wherein the group (b) of pulses include pulses having parameters to induce a stress on the cell membrane, enlarging the cell pores from the group (a) and to facilitate electrophoresis within the tissue, accelerating the loss of ions from the electroporated cells, reducing the capacity for recovery and triggering non-thermal cell death.

3. The apparatus as claimed in claim 12, wherein the group (b) of pulses include pulses having parameters to induce a stress on the cell membrane, enlarging the cell pores from the group (a) and to facilitate electrophoresis within the tissue, accelerating the loss of ions from the electroporated cells, reducing the capacity for recovery and triggering non-thermal cell death; and wherein at least some of said (b) group of pulses have parameters to trigger cell death without destruction of an extracellular matrix.

4. The apparatus as claimed in claim 1, wherein the (a) group comprises a series of M bursts of pulses, each burst having a plurality of pulses.

5. The apparatus as claimed in claim 1, wherein at least some of the (a) group comprises bipolar/biphasic pulses.

6. The apparatus as claimed in claim 1, wherein at least some of the (a) group of pulses have a field strength in the range of 350 V/cm to 1500 V/cm.

7. (canceled)

8. (canceled)

9. The apparatus as claimed in claim 1, wherein at least some of the group of pulses each have a pulse length in the range of 0.1 μs to 10 ms.

10. (canceled)

11. (canceled)

12. The apparatus as claimed in claim 1, wherein at least some of the (a) group of pulses are provided in bursts of pulses separated by time delays which are greater than time delays between individual pulses, and at least some bursts each have a duration in the range of 0.1 ms to 1000 ms.

13. The apparatus as claimed in claim 1, wherein at least some of the (a) group of pulses are provided in bursts of pulses separated by time delays which are greater than time delays between individual pulses, and at least some bursts each have a duration in the range of 0.1 ms to 5 ms.

14. The apparatus as claimed in claim 1, wherein at least some of the (a) group of pulses are provided in bursts of pulses separated by time delays which are greater than time delays between individual pulses, and the number of bursts in each cycle is in the range of 1 to 1000.

15. (canceled)

16. The apparatus as claimed in claim 1, wherein the group (b) of pulses includes pulses having a field strength in the range of 5 V/cm to 75 V/cm.

17. The apparatus as claimed in claim 1, wherein at least some of the group (b) of pulses include pulses which are square wave monophasic pulses of positive or negative polarity.

18. The apparatus as claimed in any preceding-claim 1, wherein at least some of the group (b) of pulses include at least one pulse having a length in the range of 10 ms to 1000 ms.

19. (canceled)

20. The apparatus as claimed in claim 1, wherein at least some of the group (b) of pulses include at least one pulse having a length in the range of 10 ms to 1000 ms; and wherein said length is in the range of 250 ms to 750 ms.

21. (canceled)

22. The apparatus as claimed in claim 1, wherein the total time for the treatment is in the range of 6 ms to 20 seconds.

23. The apparatus as claimed in claim 1, wherein the controller is configured to choose the value of N according to a volume of tissue to be treated.

24. (canceled)

25. The apparatus as claimed in claim 1, wherein the value of N is in the range of 2 to 10.

26. (canceled)

27. The apparatus as claimed in claim 1, wherein the (a) group has a total energised time in the range of 5 ms to 20 ms for each cycle.

28. A method of treatment of tissue in a mammal, the treatment being carried out with use of a probe having at least two electrodes and a drive circuit controlled by a controller to generate drive pulses, wherein the controller causes the drive circuit to provide pulses in:
(a) a reversible electroporation group of pulses (60) sufficient to create/open cell pores and/or disrupt cell membranes of tissue near the electrodes, wherein the apparatus at least some of the (a) group of pulses provide a field strength in the range of 100 V/cm to 2500 V/cm;
(b) a secondary group of one or more pulses (65) at least some of which have lower voltages than pulses of the group (a), wherein the group (b) of pulses includes pulses having a field strength in the range of 1 V/cm to 100 V/cm; and
(c) repetition of (a) and (b) in N cycles, N having a value of 10 to 100.

29. The method as claimed in claim 28, wherein the group (b) of pulses include pulses having parameters to induce a stress on the cell membrane, enlarging the cell pores from the group (a) and to facilitate electrophoresis within the tissue, accelerating the loss of ions from the electroporated cells, reducing the capacity for recovery and triggering non-thermal cell death.

30. The method as claimed in claim 28, wherein the group (b) of pulses include pulses having parameters to induce a stress on the cell membrane, enlarging the cell pores from the group (a) and to facilitate electrophoresis within the tissue, accelerating the loss of ions from the electroporated cells, reducing the capacity for recovery and triggering non-thermal cell death; and wherein said (b) group of pulses have parameters to trigger cell death without destruction of an extracellular matrix.
